(19) United States
(12) Patent Application Publication
(10) Pub. No.: US 2009/0275964 A1
(43) Pub. Date: Nov. 5, 2009

(54) METHOD OF ROLLING STOMACH TISSUE SO AS TO MAXIMIZE THE CONTACT SURFACE AREA OF A FOLD

(76) Inventors: Mark S. Zeiner, Mason, OH (US); Jason L. Harris, Mason, OH (US); Michael J. Stokes, Cincinnati, OH (US); Daniel E. Alesi, Lebanon, OH (US); Mark S. Ortiz, Milford, OH (US); Thomas E. Albrecht, Cincinnati, OH (US); James T. Spivey, Cincinnati, OH (US); Lawrence Crainich, Charlestown, NH (US)

Correspondence Address: WELSH & FLAXMAN LLC, 2000 DUKE STREET, SUITE 100, ALEXANDRIA, VA, 22314, US

(21) Appl. No.: 12/113,773

(22) Filed: May 1, 2008

(51) Int. Cl. A61B 17/12; A61B 17/34

(52) U.S. Cl. 606/155; 606/185

(57) ABSTRACT

A rolling instrument for rolling the outside surface of the stomach for reducing stomach volume in bariatric surgery includes an elongated tubular body that is sufficiently flexible to allow for access to the outside surface. The tubular body includes a proximal end and a distal end. The proximal end is shaped and dimensioned for use by the medical practitioner performing the medical procedure and includes a handle for manipulation of the rolling instrument. The distal end of the tubular body includes a mechanism allowing for gripping of the stomach in a manner permitting rolling thereof. A coiled retainer shaped and dimensioned is provided for application to the rolled portion of the stomach in a manner holding the rolled portion of the stomach in its rolled configuration. The coiled retainer includes a helically coiled body having a first end and a second end, the first end is pointed such that it may puncture tissue as the coiled body is rotated during its application to the stomach.

### BACKGROUND OF THE INVENTION

1. Field of the Invention

The invention relates to bariatric surgical procedures and apparatuses. More particularly, the invention relates to a method and apparatus for rolling stomach tissue in gastric reduction surgery.

2. Description of the Related Art

Obesity is a medical condition affecting more than 30% of the population in the United States. Obesity affects an individual's personal quality of life and contributes significantly to morbidity and mortality. Obese patients, i.e., individuals having a body mass index (“BMI”) greater than 30, often have a high risk of associated health problems (e.g., diabetes, hypertension and respiratory insufficiency), including early death. With this in mind, and as those skilled in the art will certainly appreciate, the monetary and physical costs associated with obesity are substantial. In fact, it is estimated the costs relating to obesity are in excess of 100 billion dollars in the United States alone. Studies have shown that conservative treatment with diet and exercise alone may be ineffective for reducing excess body weight in many patients. Bariatrics is the branch of medicine that deals with the control and treatment of obesity. A variety of surgical procedures have been developed within the bariatrics field to treat obesity. The most common currently performed procedure is the Roux-en-Y gastric bypass (RYGB). This procedure is highly complex and is commonly utilized to treat people exhibiting morbid obesity. In a RYGB procedure a small stomach pouch is separated from the remainder of the gastric cavity and attached to a resectioned portion of the small intestine. This resectioned portion of the small intestine is connected between the “smaller” gastric cavity and a distal section of small intestine allowing the passage of food therebetween. The conventional RYGB procedure requires a great deal of operative time. Because of the degree of invasiveness, post-operative recovery can be quite lengthy and painful. Still more than 100,000 RYGB procedures are performed annually in the United States alone, costing significant health care dollars.

In view of the highly invasive nature of the RYGB procedure, other less invasive procedures have been developed. These procedures include gastric banding, which constricts the stomach to form an hourglass shape. This procedure restricts the amount of food that passes from one section of the stomach to the next, thereby inducing a feeling of satiety. A band is placed around the stomach near the junction of the stomach and esophagus. The small upper stomach pouch is filled quickly, and slowly empties through the narrow outlet to produce the feeling of satiety. Other forms of bariatric surgery that have been developed to treat obesity include Fobi pouch, bilio-pancreatic diversion and gastroplasty or “stomach stapling”.

Morbid obesity is defined as being greater than 100 pounds over one's ideal body weight. For individuals in this category, RYGB, gastric banding or another of the more complex procedures may be the recommended course of treatment due to the significant health problems and mortality risks facing the individual. However, there is a growing segment of the population in the United States and elsewhere who are overweight without being considered morbidly obese. These persons may be 20-30 pounds overweight and want to lose the weight, but have not been able to succeed through diet and exercise alone. For these individuals, the risks associated with the RYGB or other complex procedures often outweigh the potential health benefits and costs. Accordingly, treatment options should involve a less invasive, lower cost solution for weight loss.

It is known to create cavity wall plications through endoscopic only procedures. However, operating solely within the interior of the gastric cavity limits the plication depth that can be achieved without cutting. Furthermore, access and visibility within the gastric and peritoneal cavities is limited in a purely endoscopic procedure as the extent of the reduction increases.

With the foregoing in mind, it is desirable to provide surgical weight loss procedures (and associated medical instruments) that are inexpensive, with few potential complications, and that provide patients with a weight loss benefit while buying time for the lifestyle changes necessary to maintain the weight loss. Further, it is desirable that the procedure be minimally invasive to the patient, allowing for a quick recovery and less scarring. The present invention provides such a procedure as associated medical instrument.

### SUMMARY OF THE INVENTION

It is, therefore, an object of the present invention to provide a rolling instrument for rolling the outside surface of the stomach for reducing stomach volume in bariatric surgery. The rolling instrument includes an elongated tubular body that is sufficiently flexible to allow for access to the outside surface. The tubular body includes a proximal end and a distal end. The proximal end is shaped and dimensioned for use by the medical practitioner performing the medical procedure and includes a handle for manipulation of the rolling instrument. The distal end of the tubular body includes a mechanism allowing for gripping of the stomach in a manner permitting rolling thereof. A coiled retainer shaped and dimensioned is provided for application to the rolled portion of the stomach in a manner holding the rolled portion of the stomach in its rolled configuration. The coiled retainer includes a helically coiled body having a first end and a second end, the first end is pointed such that it may puncture tissue as the coiled body is rotated during its application to the stomach.

It is also an object of the present invention to provide a rolling instrument wherein the tubular body includes a central lumen and a proximal port for the application of a suction mechanism used in creating a vacuum applied at the distal end of the tubular body. The distal end of the tubular body includes a series of vacuum ports formed within the surface of the tubular body. The vacuum ports maintain the central lumen of the tubular body in fluid communication with the outside surface of the stomach to allow for drawing tissue against the outer surface of the tubular body upon the application of a vacuum to the rolling instrument.

It is another object of the present invention to provide a rolling instrument wherein the rolling instrument includes an outer tube in which the operating mechanisms of the instrument are housed.

It is a further object of the present invention to provide a rolling instrument wherein the rolling instrument includes a first plication arm and a second plication arm at the distal end of the tubular body.

It is also an object of the present invention to provide a rolling instrument wherein the first plicaton arm and the second plication arm respectively includes shape memory arms wherein the shape memory arms are activated to change their shape and securely grab the stomach tissue between the first and second plication arms in a manner allowing the rolling instrument 314 to roll stomach tissue upon rotation thereof.

It is another object of the present invention to provide a rolling instrument wherein the first plication arm is statically supported at the distal end of the tubular body of the rolling instrument.

It is a further object of the present invention to provide a rolling instrument wherein the first plication arm is stationary and is fastened into a first hole formed in a wall at the distal end of the tubular body.

It is also an object of the present invention to provide a rolling instrument wherein the second plication arm includes a first end and second end, the first end is secured to a control rod that extends through the tubular body for engagement by a user at the proximal end of the rolling instrument.

It is another object of the present invention to provide a rolling instrument wherein the first end of the second plication arm includes a short aligned segment that is coupled to an offset segment oriented substantially transverse to a longitudinal axis of the first and second plication arms, such that by rotation of the second plication arm about the axis in which the short aligned segment lies, the second end of the second plication arm is moved in a circular motion moving closer and further from the first plication arm as the second plication arm is manipulated.

It is a further object of the present invention to provide a rolling instrument wherein the gripping mechanism includes shape memory arms that may be formed at the distal end of the tubular body wherein the shape memory arms are activated or extend to change their shape and securely grab the stomach tissue in a manner allowing the rolling instrument to roll stomach tissue upon rotation thereof.

It is also an object of the present invention to provide a rolling instrument wherein the gripping mechanism includes a retractable spike system.

It is another object of the present invention to provide a rolling instrument wherein the retractable spike system is composed of a series of selectively extendable spikes.

It is a further object of the present invention to provide a rolling instrument wherein distal end of the tubular body includes a series of recesses shaped and dimensioned to provide a housing for the spikes prior to actuation.

It is also an object of the present invention to provide a rolling instrument wherein the spikes are L-shaped spikes and include a first leg and a second leg held at approximately a 90° angle.

It is another object of the present invention to provide a rolling instrument wherein the spikes are made from a shape memory material or superelastic material.

It is a further object of the present invention to provide a rolling instrument wherein a spike driver is secured to the spikes for actuation thereof.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a cross sectional view of the stomach prior to gastric reduction surgery in accordance with the present invention.

FIG. 2 is a cross sectional view of the stomach after rolling of the outer surface of the stomach so as to reduce stomach volume in accordance with the present invention.

FIG. 3 is a perspective schematic view showing utilization of a rolling instrument in accordance with a preferred embodiment of the present invention.

FIG. 4 is a perspective view of the distal end of the rolling instrument shown with reference to FIG. 3.

FIGS. 5, 6, 7 and 8 show the steps associated with forming a roll along the outer surface of the stomach with the rolling instrument shown with reference to FIGS. 3 and 4.

FIGS. 9 and 10 are perspective views of a coiled retainer for use in accordance with the present invention.

FIG. 11 shows a schematic of an alternate embodiment of a rolling instrument in accordance with the present invention.

FIG. 12 is a perspective view of the rolling instrument shown with reference to FIG. 11.

FIGS. 13, 14, 15, 16 and 17 show the steps associated with rolling of the stomach wall with the rolling instrument shown with reference to FIGS. 11 and 12.

FIG. 18 is a schematic showing a rolling instrument in accordance with yet another embodiment.

FIG. 19 shows Nitinol arms in their deployed configurations (FIG. 18 shows the spikes in the undeployed configuration).

FIGS. 20, 21, 22, and 23 show use of the rolling instrument disclosed with reference to FIGS. 18 and 19.

FIG. 24 is a schematic showing a rolling instrument in accordance with yet another embodiment.

FIGS. 25 and 26 show spikes in their respective deployed and undeployed configurations.

FIG. 27 is an exploded perspective view showing the rolling instrument of FIG. 24 with the spikes deployed and

FIG. 28 shows the same rolling instrument with the spikes in their undeployed configuration

FIGS. 29, 30 and 31 show use of the rolling instrument disclosed with reference to FIGS. 24-28.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

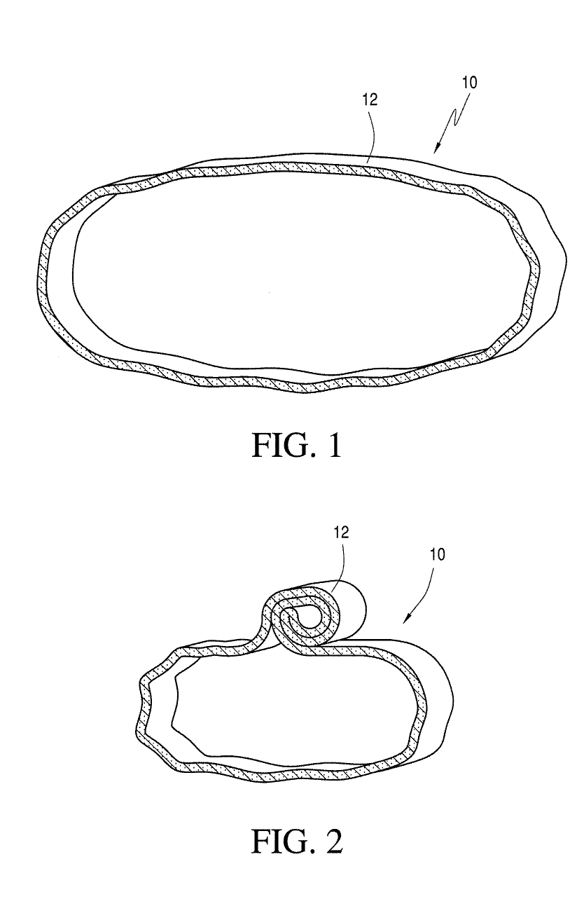

In accordance with the present invention, and with reference to FIGS. 1 and 2 and the various embodiments discloses herein, the present invention relates to a method and apparatus for reducing stomach volume in bariatric surgery by rolling the outside surface 12 of the stomach 10 and retaining it in that position to reduce the volume of the gastric cavity. While the present invention discloses rolling of the stomach along the outer surface of the stomach, it is contemplated internal rolling of the stomach wall may also be achieved. Whether rolling is performed on the internal surface or the external surface of the stomach, it may be performed by either endoscopic (that is, via a natural orifice, for example, transorally) or laparoscopic (that is, via a surgical incision through a body wall) techniques. Rolling of the outside surface provides an improved angle of approach for ultimately accessing the desired surfaces of the stomach and provides the medical practitioner with substantially more working space then if the procedure were performed within the gastric cavity. By reducing stomach volume in this manner, satiety is achieved with less food intake.

Briefly, the method is achieved by the provision of a gripping mechanism vacuum system that allows for rolling of the outside surface of the stomach and a method for retention of the stomach in the rolled configuration. The rolling of the stomach wall in accordance with the various embodiments of the present invention creates a serosa-to-serosa surface interaction that is highly desirable in gastric reduction procedures. In particular, it has been found that when the serosal surfaces of the stomach are brought together a tissue bond is formed over time which securely holds the stomach in its folded configuration in a manner providing for long term gastric reduction without the need for additional fastening elements.

More particularly, and with reference to FIGS. 1 and 2, a schematic showing an untreated stomach 10 and the same stomach 10 after rolling in accordance with the present invention is presented. As seen when the untreated stomach 10 (FIG. 1) is compared with the treated stomach 10 (FIG. 2), the volume of the internal gastric cavity of the stomach 10 is greatly reduced by simply rolling and retaining a portion of the outside surface 12 of the stomach 10 in accordance with the present invention as is discussed below in greater detail.

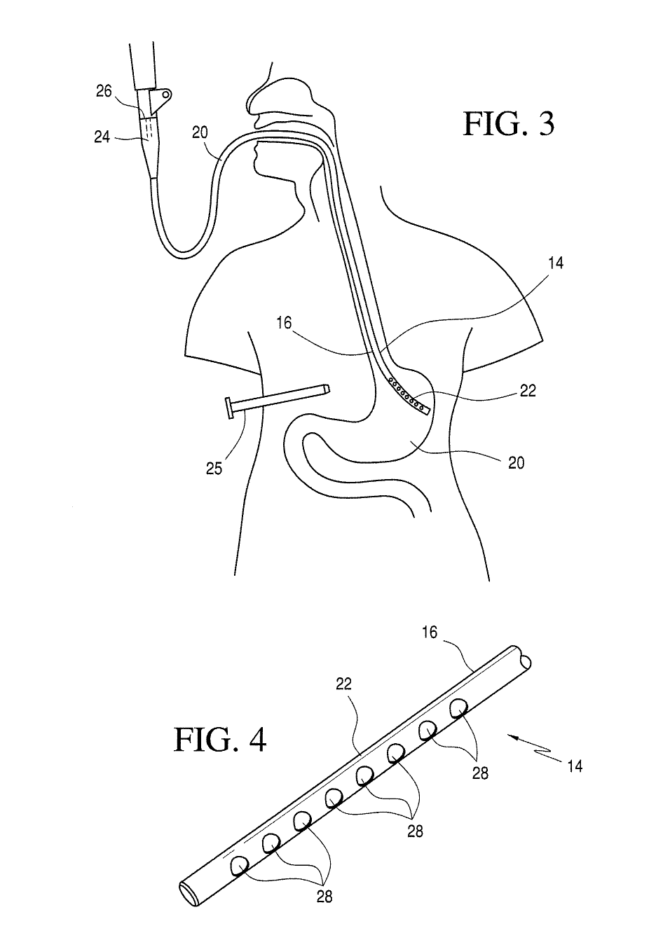

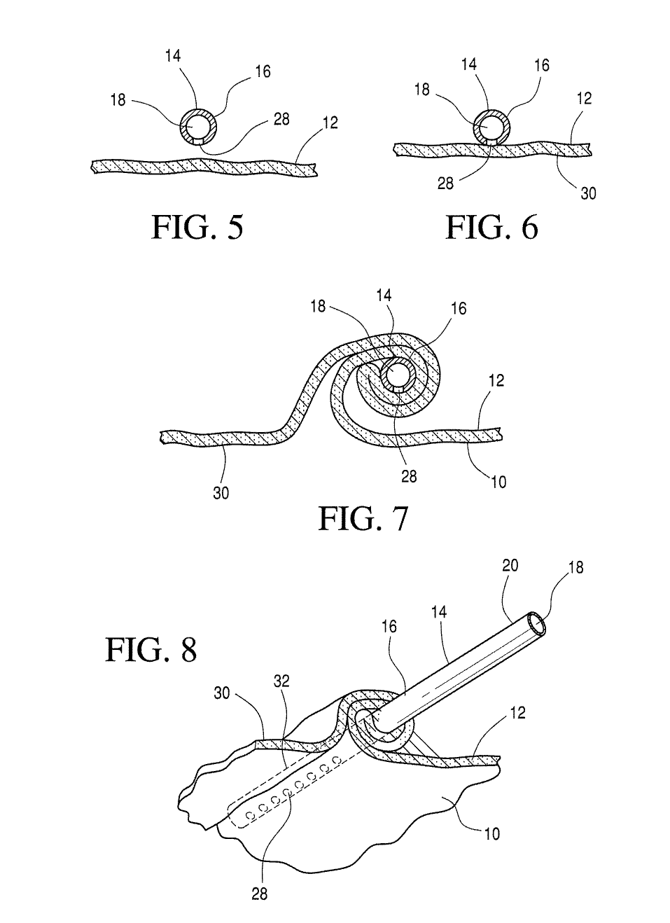

Referring to FIGS. 3 to 10, a preferred embodiment of a rolling instrument 14 for use in rolling the stomach 10 in accordance with a preferred embodiment of the present invention is disclosed. The rolling instrument 14 includes an elongated tubular body 16 with a central lumen 18. The tubular body 16 is sufficiently flexible for intubation and to allow the tubular body 16 to lie upon and follow the tissue contour of the outside surface 12 of the stomach 10. Although the rolling instrument 14 is shown in the stomach 10 it could be inserted through a trocar 25 to be used on the outside surface 12 of the stomach 10. The tubular body 16 includes a proximal end 20 and a distal end 22. The proximal end 20 is shaped and dimensioned for use by the medical practitioner performing the medical procedure. As such, the proximal end 20 includes a handle 24 for manipulation of the rolling instrument 14 and a proximal port 26 for the application of a suction mechanism used in creating the vacuum applied at the distal end 22 of the tubular body 16. In particular, the distal end 22 of the tubular body 16 includes a gripping mechanism composed of a series of vacuum ports 28 formed within the surface of the tubular body 16. The vacuum ports 28 maintain the central lumen 18 of the tubular body 16 in fluid communication with the outside surface 12 of the stomach 10 to allow for drawing tissue against the outer surface of the tubular body 16 upon the application of a vacuum to the rolling instrument 14.

In practice, the rolling instrument 14 is positioned adjacent the outside surface 12 of the stomach 10 or the inside surface of stomach 10 and a vacuum is applied. The applied vacuum is sufficient to draw the outside surface 12 of the stomach 10 into contact with the distal end 22 of the tubular body 16. Once the outside surface 12 is securely held against the distal end 22 of the tubular body 16 under the control of the applied vacuum, the medical practitioner rotates the tubular body 16 about its longitudinal axis. Rotation is preferably achieved by rotation of the entire medical instrument, although it is contemplated other actuation mechanisms may be implemented within the spirit of the present invention. This rotation causes the stomach wall 30 to wrap around the distal end 22 of the tubular body 16 creating a roll in the outside surface 12 of the stomach 10 at a location where the rolling instrument 14 is applied.

Once the stomach wall 30 is rolled about the distal end 22 of the tubular body 16 of the rolling instrument 14, the stomach tissue is retained in its rolled configuration. In accordance with a preferred embodiment, a coiled retainer 32 is applied to the rolled portion of the stomach 10 in a manner holding the rolled portion of the stomach 10 in its rolled configuration. The coiled retainer 32 includes a helically coiled body 34 having a first end 36 and a second end 38. The first end 36 is pointed such that it may puncture tissue as the coiled body 34 is rotated during its application to the stomach 10. In use, the coiled retainer 32 is oriented with the first end 36 thereof positioned at one end of the coiled section of the stomach 10 the medical practitioner wishes to retain in a rolled configuration. The coiled retainer 32 is then retained with the pointed first end 36 passing through the tissue. As rotation of the coiled retainer 32 is continued, the coiled retainer 32 will move along the length of the rolled section of tissue with the first end 36 thereof continuing to rotate around the rolled section and puncturing tissue, while drawing the remainder of the coiled retainer 32 about the rolled section of the stomach 10.

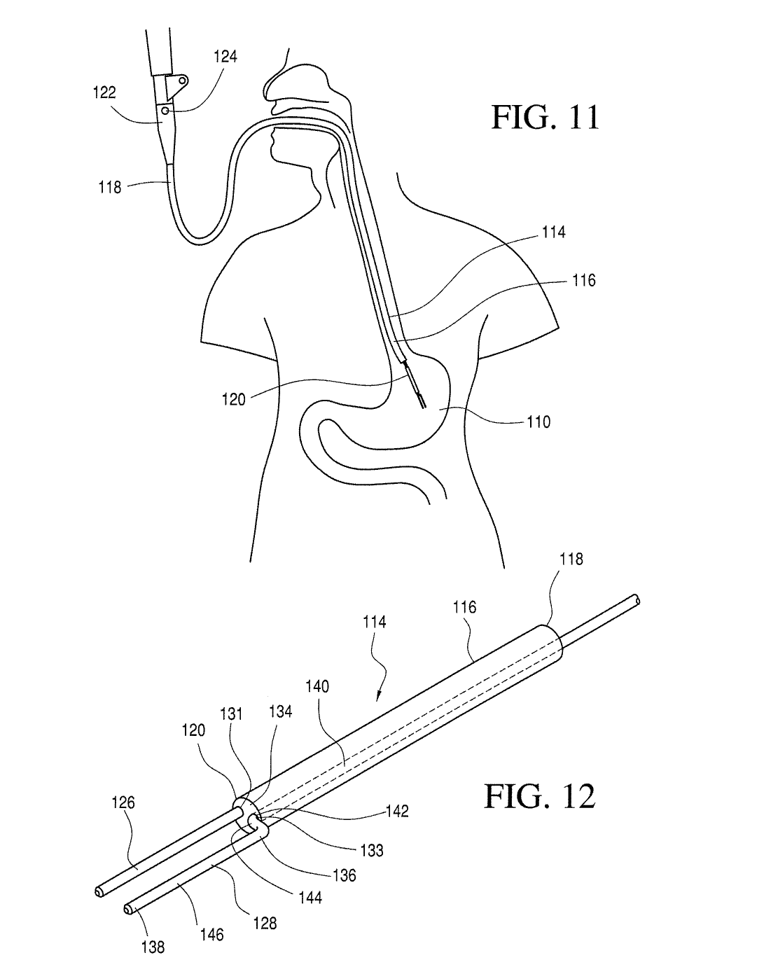

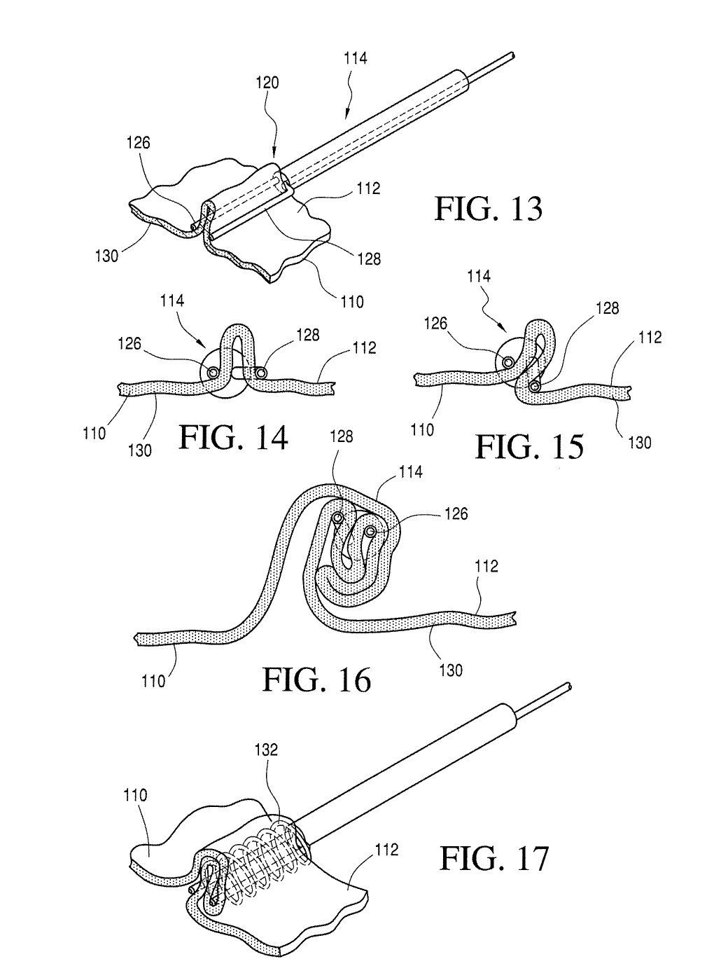

In accordance with an alternate embodiment, and with reference to FIGS. 11 to 17, a different medical rolling instrument 14 may be employed in creating a rolled section in accordance with a preferred embodiment of the present invention. The rolling instrument 14 includes an elongated tubular body 116 that is sufficiently flexible to allow the tubular body 16 to lie upon and follow the tissue contour of the outside surface 112 of the stomach 110. Where the present rolling instrument is intended for use outside of the stomach through a cannula that provides access to the body cavity, the rolling instrument will include an outer tube in which the operating mechanisms of the instrument are housed. Where the present medical instrument is to be used internally, it is provided with a body that is as short as possible for easy insertion through the esophagus and would be provided with a reduced diameter, a flexible shaft and a handle mechanism allowing it to be extended outside the mouth of the patient.

In accordance with the embodiment shown with reference to FIGS. 11 to 17, the tubular body 116 includes a proximal end 118 and a distal end 120. The proximal end 118 is shaped and dimensioned for use by the medical practitioner performing the medical procedure. As such, the proximal end 118 includes a handle 122 for manipulation of the rolling instrument 114 and control knobs 124 for actuation of the distal end 120 of the tubular body 116 and discussed below in greater.

The distal end 120 of the tubular body 116 includes a gripping mechanism composed of a first plication arm 126 and a second plication arm 128 used in grabbing the outside surface 112 of the stomach 110 and rotating the tissue to roll it into a desired configuration resulting in the reduction of the volume of the stomach cavity. The first plication arm 126 is statically supported at the distal end 120 of the tubular body 116 of the rolling instrument 114. In accordance with a preferred embodiment, the first plication arm 126 is stationary and is fastened into one of the first and second holes 131, 133 formed in the wall 134 at the distal end 120 of the tubular body 116 of the present rolling instrument 114. The second plication arm 128 has a jog form in it, that is, a lateral offset, and it fits through the other hole 133 in the bushing. In particular, the second plication arm 128 includes a first end 136 and second end 138. The first end 136 is secured to the control rod 140 that extends through the tubular body 116 for engagement by a user at the proximal end 118 of the present rolling instrument 114. The first end 136 includes a short aligned segment 142 that is coupled to an offset segment 144 oriented substantially transverse to a longitudinal axis of the first and second plication arms 126, 128. Finally, the offset segment 144 leads to an aligned engagement section 146 extending to the second end 138 that is substantially aligned with the longitudinal axis of the first and second plication arms 126, 128. As such, by rotation the second plication arm 128 about the axis in which the short aligned segment 142 lies, the aligned engagement section 146 is moved in a circular motion moving closer and further from the first plication arm 126 as the second plication arm 128 is manipulated.

In particular, rotation of the control rod 140 of the rolling instrument 114 causes the second plication arm 128 to be moved toward the first plication arm 126 in a controlled manner. As shown in the various Figures, the first plication arm 126 and the second plication arm 128 may be oriented in close approximation (or actually touch) so that they fit through a cannula or esophagus for insertion of the rolling instrument 114 in a controlled and convenient manner.

In practice, the rolling instrument 114 is positioned adjacent the outside surface 112 of the stomach 110 and the second plication arm 128 is moved toward the first plication arm 126 such that a portion of the outside surface 112 of the stomach 110 is securely held between the first and second plication arms 126, 128. Once the outside surface 112 of the stomach 110 is securely held between the first and second plication arms 126, 128 at the distal end 120 of the rolling instrument 114, the medical practitioner rotates the rolling instrument 114 about its longitudinal axis. This rotation causes the stomach wall to wrap around the distal end 120 of the rolling instrument 114 creating a roll in the outside surface 112 of the stomach 110 at a location where the rolling instrument 114 was applied.

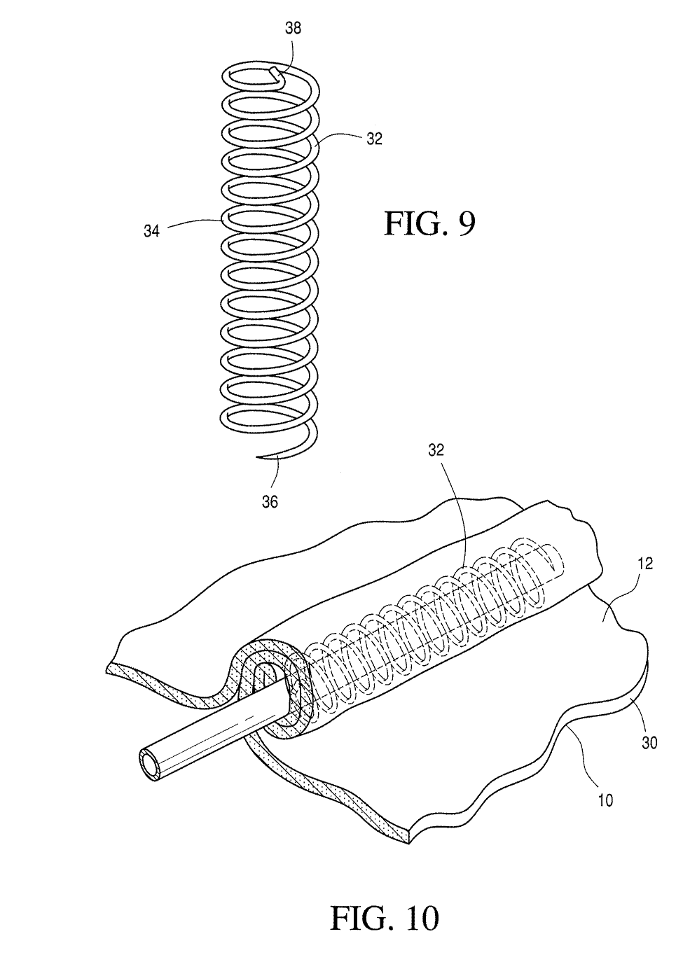

Once the stomach wall 130 is rolled about the distal end 120 of the rolling instrument 114 creating a desired reduction in the volume of the stomach cavity, the stomach tissue is retained in its rolled configuration. In accordance with a preferred embodiment, and as discussed above in accordance with the embodiment shown with reference to FIGS. 3 to 10, a coiled retainer 132 is applied to the rolled portion of the stomach 110 in a manner holding the rolled portion of the stomach 110 in its rolled configuration. As with the embodiment discussed above with reference to FIGS. 9 and 10, the coiled retainer 132 includes a helically coiled body having a first end and a second end. The first end is pointed such that it may puncture tissue as the coiled body is rotated during its application to the stomach. In use, the coiled retainer is oriented with the first end thereof positioned at one end of the coiled section the medical practitioner wishes to retain in a rolled configuration. The coiled retainer is then retained with the pointed first end passing through the tissue. As rotation of the coiled retainer is continued, the coiled retainer will move along the length of the rolled section with the first end thereof continuing to rotate around the rolled section and puncturing tissue, while drawing the remainder of the coiled retainer about the rolled section of the stomach.

In accordance with alternate embodiment, other fastening structures may be employed within the spirit of the present invention. The fastening structures may be suture, staples, clips, t-tags, adhesive or other mechanism that are capable of holding the tissue in its rolled configuration. The fastening structures penetrate the gastric layers and hold them in place either permanently or long enough for the tissue to adhere. The fastening structures may be bioabsorbable.

Once the rolled tissue is securely held in position, the second plication arm 128 is rotated to release the tissue from between the first and second plication arms 126, 128, and the rolling instrument 114 may be removed from its position within the stomach 110.

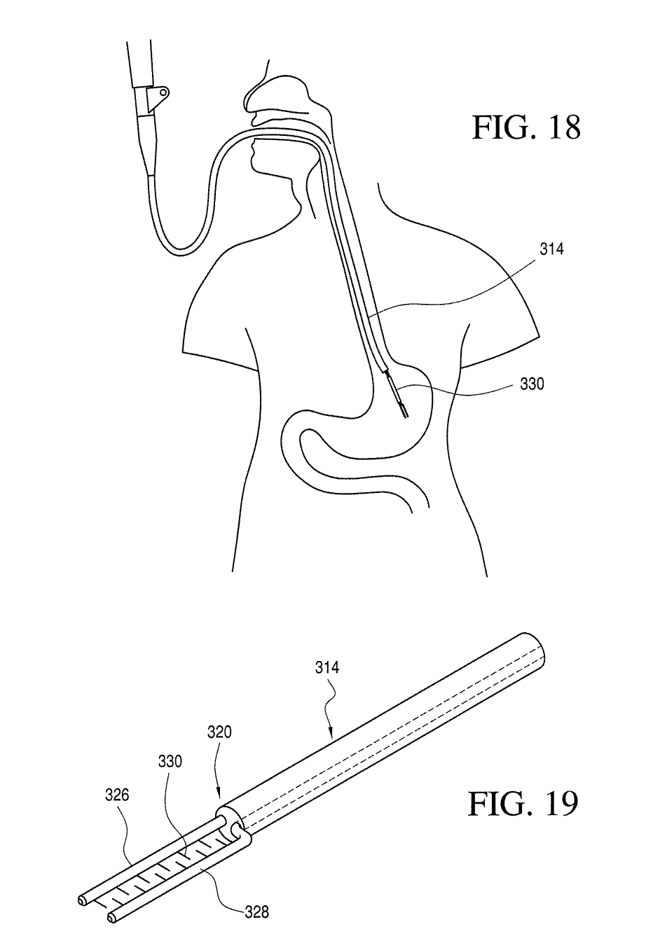

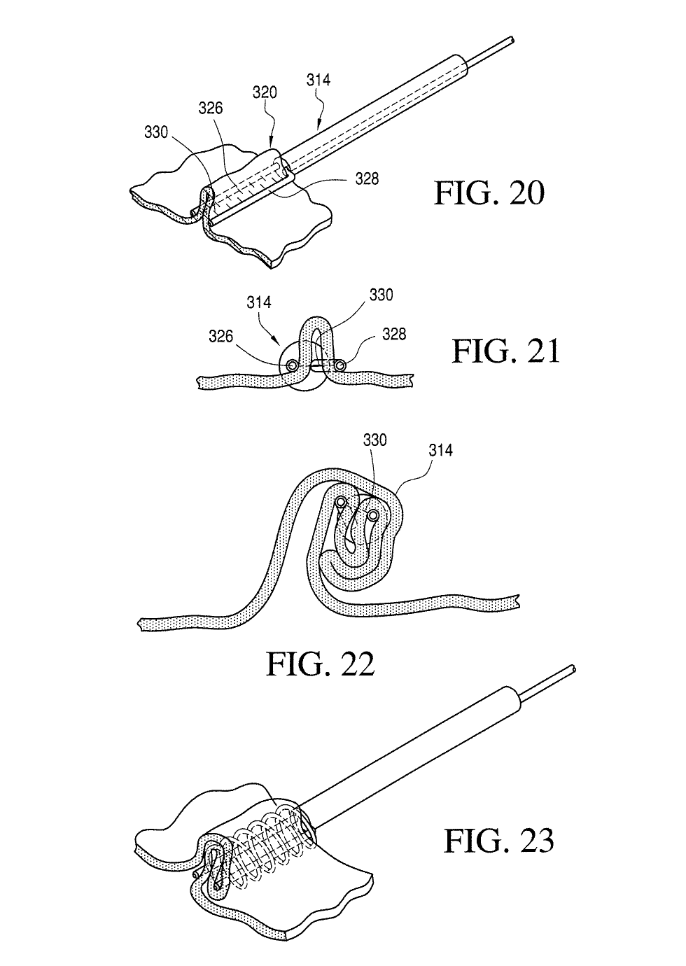

Similar, and with reference to FIGS. 18 to 23, the gripping mechanism at the distal end 320 of the rolling instrument 314 may be formed with first and second plication arms 326, 328 as shown with reference to the embodiment shown in FIGS. 11 to 18. However, and rather than moving the plication arms 326, 328 for securing the stomach tissue therebetween, a plurality of laterally extending Nitinol arms 330 may be formed along the first and second plication arms 326, 328 wherein the Nitinol arms 330 are activated to change their shape and securely grab the stomach tissue between the first and second plication arms 326, 328 in a manner allowing the rolling instrument 314 to roll stomach tissue upon rotation thereof.

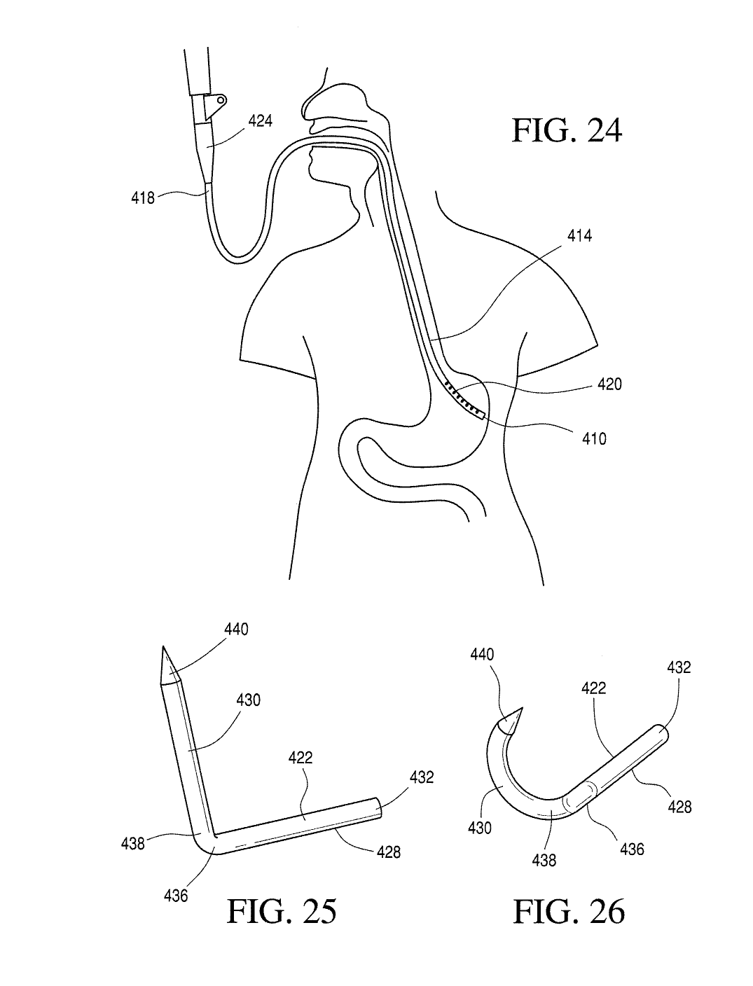

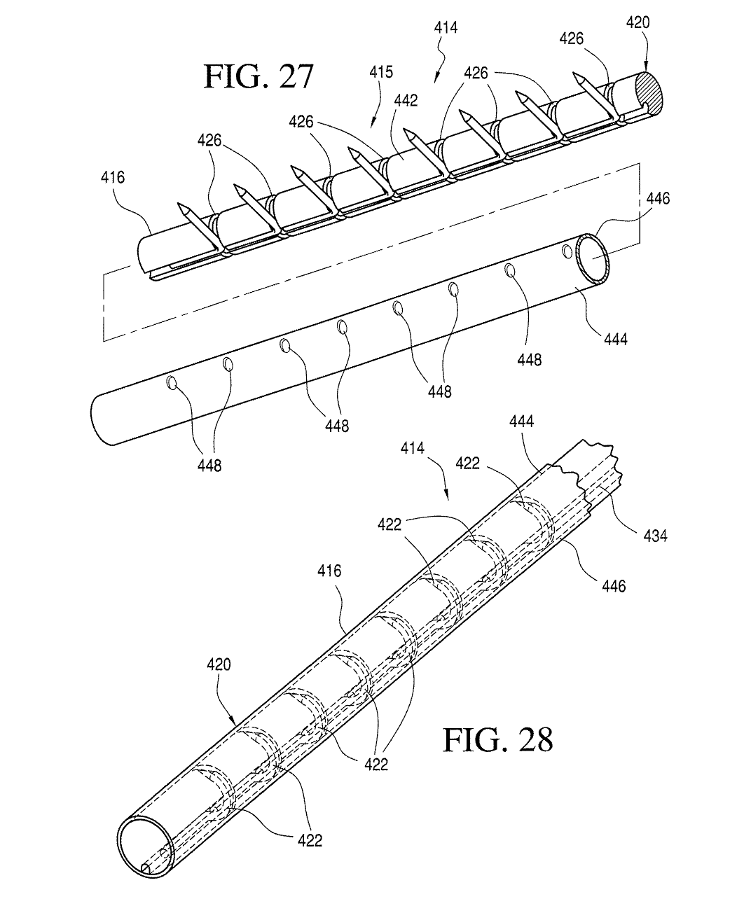

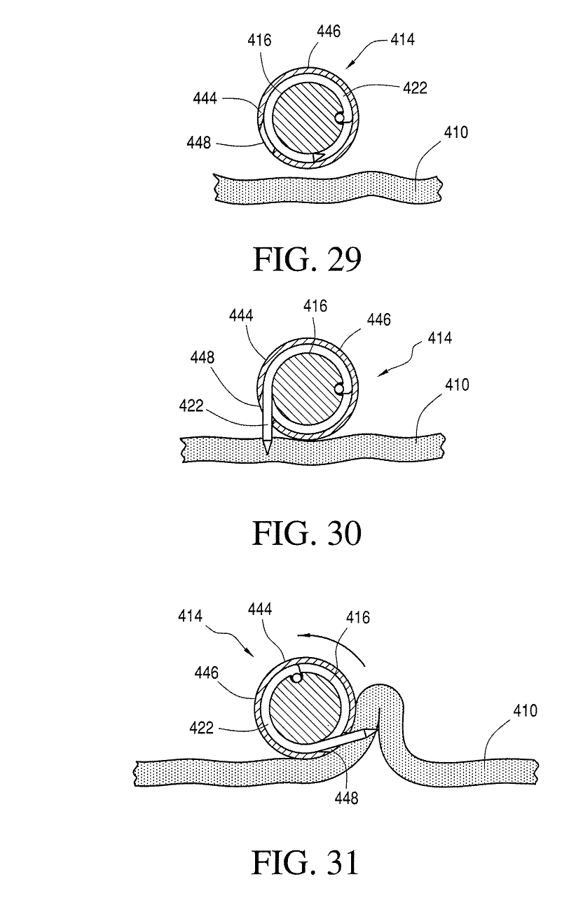

In accordance with an alternate embodiment and with reference to FIGS. 24 to 31, a rolling instrument 414 with a gripping mechanism composed of a retractable spike system for rolling stomach tissue is disclosed. As with the prior embodiment, the retractable spike system 415 allows medical practitioners to reduce stomach volume by rolling the outside surface 412 of stomach 410 and retaining it in that position to reduce the volume of the inside of the stomach 410. Briefly, the rolling instrument 414 includes a tubular body 416 having a proximal end 418 and a distal end 420. The distal end 420 of the tubular body 416 is provided with a series of spikes 422 that snag stomach tissue. Once the spikes 422 are extended in a manner discussed below in greater detail, the rolling instrument 414 is rotated to gather tissue and consequently reduce the volume of the stomach cavity. Once the stomach tissue is rolled, various known fastening mechanisms may be utilized to securely hold the stomach in the desired configuration.

Referring to FIGS. 24 to 31, the retractable spike system 415 includes an elongated tubular body (or shaft) 416. The tubular body 416 is sufficiently flexible to allow the tubular body 16 to lie upon and follow the tissue contour of the outside stomach surface. The tubular body 416 includes a proximal end 418 and a distal end 420. The proximal end 418 includes a handle mechanism 424 shaped and dimensioned for use by the medical practitioner performing the medical procedure. As such, the proximal end 418 includes a handle mechanism 424 for manipulation of the retractable spike system 415 in accordance with the present invention.

The distal end 420 of the tubular body 416 includes a series of recesses 426 along the outer surface thereof. The recesses 426 provide a housing for the spikes 422 prior to actuation. In fact, the recesses 426 are shaped to house the spikes 422 such that they substantially conforming to the outer surface of the tubular body 416 prior to actuation thereof in accordance with the present invention.

In accordance with a preferred embodiment of the present invention, the spikes 422 are L-shaped spikes. The L-shaped spikes 422 include a first leg 428 and a second leg 430 held at approximately a 90° angle. The first leg 428 includes a first end 432 secured to a spike driver 434 coupled to each of the spikes 422 for controlling rotation thereof between a storage position with the spikes 422 held within the recesses 426 and an extended position with the spikes 422 rotated outwardly for engagement with tissue. The first leg 428 also includes a second end 436 that is secured to the first end 438 of the second leg 430. The second end 440 of the second leg 430 is pointed for piercing into the stomach 410 to hold but not necessarily pierce through the stomach tissue. In addition, and as will be appreciate the second leg 430 is curved to conform with the shape of the recesses 426 and the tubular body 416. However, the second leg 430 is preferably made from shape memory material allowing it to extend to an elongated, straightened configuration upon deployment.

In accordance with a preferred embodiment, the spike 422 is made from Nitinol or other material that has shape memory or spring qualities such that the spike 422 can be manipulated from a rolled position in which it is stored within the recesses 426 formed along the distal end 420 of the tubular body 416 and an L-shaped orientation for attachment to the tissue.

As briefly mentioned above, the tubular body 416 includes an array of recesses 426 at its distal end 420 from which the plurality of spikes 422 extends in a predetermined manner. Secured to the various spikes 422 is a spike driver 434. Rotation of the spike driver 434 causes the spikes 422 to extend from the outer wall 442 of the tubular body 416 in a controlled manner for engagement with tissue in a desired manner. In particular, the spike driver 434 is secured to the various spikes 422 such that upon rotation of the spike driver 434 the spikes 422 are rotated from the storage position to an extended position.

A cover 444 composed of a tubular member 446 is secured over the tubular body 416 for controlled release of the various spikes. The tubular member 446 includes a series of apertures 448 in alignment with the recesses 426 such that the second legs 430 of the spikes 422 may extend therethrough when deployment is desired.

In accordance with a preferred embodiment, the present rolling instrument 414 is inserted into the body cavity through a cannula with the spikes 422 retracted within the recesses 426 along the outer wall 442 of the tubular body 416. The apertures 448 of the cover 444 are then aligned with the second legs 430 and the spike driver 434 is then rotated causing the spikes 422 to extend. The spikes 422 are then forced to partially pierce the stomach tissue. The entire rolling instrument 414 is then rotated and the spikes 422 pull the tissue and roll it radially. Referring to FIG. 31, the stomach 410 is shown with the spikes 422 extended into the tissue and firmly embedded into the tissue with a complete roll thereof. Of course, the roll could be more or less as desired. When the desired stomach reduction is achieved, the rolled tissue is fastened so that is does not unravel. As briefly discussed above, fastening may be achieved by staples, clips, sutures, t-tags or the like.

After the roll is fastened by staples, clips, sutures, t-tags or other mechanism, the spikes are withdrawn into the outer tube and the device is withdrawn from inside the roll and out of the body.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used system is obtained and if necessary cleaned. The system can then be sterilized. In one sterilization technique, the system is placed in a closed and sealed container, such as a plastic or TYVEK bag. The container and system are then placed in a field of radiation that can penetrate the container, such as gamma radiation, x-rays, or high-energy electrons. The radiation kills bacteria on the system and in the container. The sterilized system can then be stored in the sterile container. The sealed container keeps the system sterile until it is opened in the medical facility.

It is preferred that the device is sterilized. This can be done by any number of ways known to those skilled in the art including beta or gamma radiation, ethylene oxide, or steam.

While the preferred embodiments have been shown and described, it will be understood that there is no intent to limit the invention by such disclosure, but rather, is intended to cover all modifications and alternate constructions falling within the spirit and scope of the invention.

1. A rolling instrument for rolling the outside surface of the stomach for reducing stomach volume in bariatric surgery, comprising:
an elongated tubular body that is sufficiently flexible to allow for access to the outside surface, the tubular body includes a proximal end and a distal end, the proximal end is shaped and dimensioned for use by a medical practitioner performing a medical procedure and includes a handle for manipulation of the rolling instrument; the distal end of the tubular body includes a mechanism allowing for gripping of the stomach in a manner permitting rolling thereof;
a coiled retainer shaped and dimensioned for application to a rolled portion of the stomach in a manner holding the rolled portion of the stomach in its rolled configuration, the coiled retainer includes a helically coiled body having a first end and a second end, the first end is pointed such that it may puncture tissue as the coiled body is rotated during the application of the coiled body to the stomach.

2. The rolling instrument according to claim 1, wherein the tubular body includes a central lumen and a proximal port for the application of a suction mechanism used in creating a vacuum applied at the distal end of the tubular body; the distal end of the tubular body includes a series of vacuum ports formed within a surface of the tubular body, the vacuum ports maintain the central lumen of the tubular body in fluid communication with the outside surface of the stomach to allow for drawing tissue against an outer surface of the tubular body upon the application of a vacuum to the rolling instrument.

3. The rolling instrument according to claim 1, wherein the rolling instrument include an outer tube.

4. The rolling instrument according to claim 1, wherein the rolling instrument includes a first plication arm and a second plication arm at the distal end of the tubular body.

5. The rolling instrument according to claim 4, wherein the first plication arm and the second plication arm respectively includes shape memory arms wherein the shape memory arms are activated to change their shape and securely grab stomach tissue between the first and second plication arms in a manner allowing the rolling instrument to roll stomach tissue upon rotation thereof.

6. The rolling instrument according to claim 4, wherein the first plication arm is statically supported at the distal end of the tubular body of the rolling instrument.

7. The rolling instrument according to claim 6, wherein the first plication arm is stationary and is fastened into a first hole formed in a wall at the distal end of the tubular body.

8. The rolling instrument according to claim 7, wherein the second plication arm includes a first end and a second end, the first end is secured to a control rod that extends through the tubular body for engagement by a user at the proximal end of the rolling instrument.

9. The rolling instrument according to claim 8, wherein the first end of the second plication arm includes a short aligned segment that is coupled to an offset segment oriented substantially transverse to a longitudinal axis of the first and second plication arms, such that by rotation of the second plication arm about an axis in which the short aligned segment lies, the second end of the second plication arm is moved in a circular motion moving closer and further from the first plication arm as the second plication arm is manipulated.

10. The rolling instrument according to claim 1, wherein the mechanism allowing for gripping includes shape memory arms may be formed at the distal end of the tubular body wherein the shape memory arms are activated to change their shape and securely grab stomach tissue in a manner allowing the rolling instrument to roll stomach tissue upon rotation thereof.

11. The rolling instrument according to claim 1, wherein the mechanism allowing for gripping includes a retractable spike system.

12. The rolling instrument according to claim 11, wherein the retractable spike system is composed of a series of selectively extendable spikes.

13. The rolling instrument according to claim 12, wherein the distal end of the tubular body includes a series of recesses shaped and dimensioned to provide a housing for the spikes prior to actuation.

14. The rolling instrument according to claim 13, wherein the spikes are L-shaped spikes and include a first leg and a second leg held at approximately a 90° angle.

15. The rolling instrument according to claim 12, wherein the spikes are made from a shape memory material or superelastic material.

16. The rolling instrument according to claim 12, wherein a spike driver is secured to the spikes for actuation thereof.
